(19) United States
(12) Patent Application Publication
(10) Pub. No.: US 2023/0233725 A1
(43) Pub. Date: Jul. 27, 2023

(54) A-ROMA-MAT

(71) Applicant: Keith Johnson, Dallas, TX (US)

(72) Inventor: Keith Johnson, Dallas, TX (US)

(21) Appl. No.: 18/092,048

(22) Filed: Dec. 30, 2022

Related U.S. Application Data
(60) Provisional application No. 63/361,500, filed on Dec. 30, 2021.

(51) Int. Cl. A61L 9/012; A61L 9/12; A61L 9/04

(52) U.S. Cl. CPC A61L 9/012; A61L 9/12; A61L 9/048

(57) ABSTRACT

A mat which generates aromatic fumes when stepped on,

### TECHNICAL FIELD OF THE INVENTION

The present invention relates, in general, to mats, and, more particularly, to scent dispersing mats.

### BACKGROUND OF THE INVENTION

There are many different applications for floor mats of various kinds. Floor mats are especially common in the bathrooms of non-residential buildings in the form of floor mats disposed under a hand dryer or urinal. Such floor mats are offered as non-slip surfaces to ensure that a user does not slip on any liquids that may have come in contact with the floor mat during use of a hand dryer or urinal.

One problem common to traditional bathroom floor mats is that the floor mats often fail to eliminate the undesirable odors associated with bathroom use. Floor mats for use with urinals and hand-dryers are especially susceptible to undesired odors as the floor mats are intended to shield the bathroom floor surface from such odorous liquids, especially urine. In some traditional floor mats, the urine or other odorous liquid is merely deposited on the floor mat, causing the odor coming from the floor mat to worsen significantly over time. Another problem associated with such floor mats is that a user who steps on the floor mat may unintentionally track any moisture or dirt found on the floor mat to another location exterior to the bathroom, causing additional odors and more areas in need of cleaning.

Floor mats serve several functions and can be found in various locations. Many automobiles have floor mats that help keep the carpet beneath the mat protected from dirt and moisture, which might otherwise be tracked into the automobile by the driver and passengers. Floor mats can often be found at the ingress points of homes and offices. Similar to automobile floor mats, these doormats usually help ensure that dirt and moisture is not brought within a premises. Floor mats also appear in bathrooms and help protect against both slipping and the spread of germs. In practice, bathroom floor mats may be found on the bathroom floor, within a bathtub, or in a shower stall, Though floor mats are very prevalent in modern life, there are several ways in which modem floor mats may be improved to provide significant advantages.

### SUMMARY OF THE INVENTION

This, and other, objects are achieved by a mat which generates aromatic fumes when stepped on.

The mat can be used in a multitude of locations, both public and private, such as kitchens, bathrooms, workrooms, storage rooms, gyms, Locke rooms and many more. The aromatic fumes can be continuously generated and increased when the mat is stepped on, or can be generated only when the mat is stepped on, whatever is suitable and desired.

The mat can also serve as a water catcher, and the fumes can be air freshener.

### BRIEF DESCRIPTION OF THE DRAWING FIGURE

FIG. 1 shows a top plan view of a mat embodying the present invention.

FIG. 2 shows an alternative form of the mat.

FIG. 3 is a side elevational view of the mat.

FIG. 4 shows the mat in use.

FIG. 5 is another view of the mat.

### DETAILED DESCRIPTION OF THE PREFERRED EMBODIMENT OF THE INVENTION

Other objects, features and advantages of the invention will become apparent from a consideration of the following detailed description and the accompanying drawings.

Referring to the figures, it can be understood that the present invention is embodied in a mat 10 which can be used in various locations and which includes a body 20 having a first surface 20 that is a top surface when the mat is in use and a second surface 30 that is a bottom surface when the mat is in use. The two surfaces define an internal volume 40 therebetween. A system of aromatic fumes is located in volume 40 and incudes a system of exhaust holes, such as hole 50, fluidically connected to a cylinder 60 which contains the fluid that is the source of fumes F that are emitted from the mat when the mat is stepped on as indicated in FIG. 4. The fluidic connection between the exhaust holes and the source can be any suitable connection and is indicated in FIG. 3 by wavy arrows.

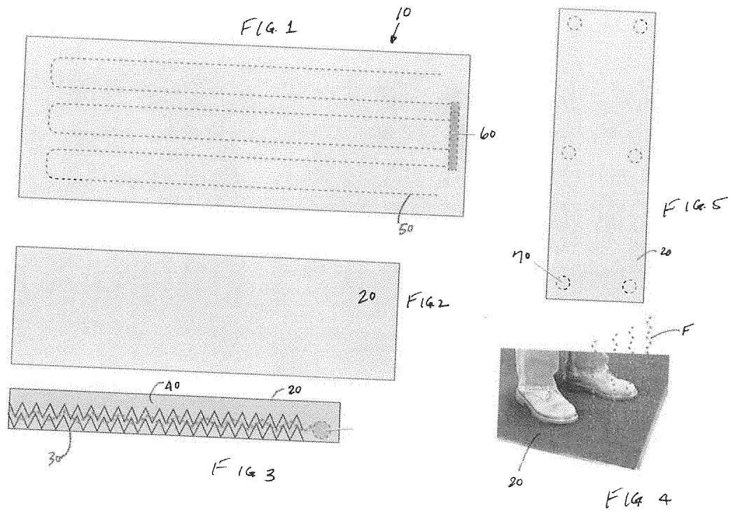

As can be understood by comparing FIGS. 1 and 2, the mat can be of various sizes. Mat 20 is held in place by suction cups, such as cup 70, located on the bottom surface of the mat.

It is understood that while certain forms of the present invention have been illustrated and described herein, it is not to be limited to the specific forms or arrangements of parts described and shown.

1. This Flooring Mat Invention To, have vented top sections of the mat with special designed vent holes at top & bottom of the mat. “Vented for aroma sent, spills or wet conditions,

2. This Flooring Mat Invention To, be design with a multiple “suction cup Bottom, an arrow shape grip pointed section on each corner end of the mat, at Bottom. “For secure placement on hard flooring or carpeted flooring,

3. This Flooring Mat Invention To, have A design that will allow the installment of, a A-ROMA, scented insertable lining/cartage that will install in the center section of the floor mat, with a fibric & gel scented aroma, fragrance material section. “Multiple scents to choose from for home, business, auto, & pet areas.
